(19) United States
(12) Patent Application Publication
(10) Pub. No.: US 2013/0131112 A1
(43) Pub. Date: May 23, 2013

(54) SALTS OF 3-(4-AMINO-1-OXO-1,3-DIHYDRO-ISOINDOL-2-YL)PIPERIDINE-2,6-DIONE AND DERIVATIVES THEREOF, OR POLYMORPHS OF SALTS, PROCESS FOR PREPARING SAME AND USE THEREOF

(71) Applicants: TIANJIN HEMAY BIO-TECH CO., LTD, Tianjin (CN); TIANJIN MICHELE SCI-TECH DEVELOPMENT C, Tianjin (CN)

(72) Inventor: Hesheng Zhang, Tianjin (CN)

(73) Assignees: TIANJIN MICHELE SCI-TECH DEVELOPMENT CO., LTD, Tianjin (CN); TIANJIN HEMAY BIO-TECH CO., LTD, Tianjin (CN)

(21) Appl. No.: 13/675,767

(22) Filed: Nov. 13, 2012

Related U.S. Application Data
(62) Division of application No. 12/880,983, filed on Sep. 13, 2010, now abandoned.
Application No. 12/880,983 is a continuation of application No. PCT/CN2009/000258, filed on Mar. 11, 2009.

(30) Foreign Application Priority Data
Mar. 13, 2008 (CN) 200810052422.8

(51) Int. Cl. C07D 401/04

(52) U.S. Cl. CPC C07D 401/04
USPC 514/323; 546/201

(57) ABSTRACT

The present invention provides a pharmaceutically acceptable strong acid salt of 3-(4-amino-1-oxo-1,3-dihydro-isoindol-2-yl)piperidine-2,6-dione, a solvate thereof, a process for preparing the same and use in the preparation of a medicament for treating diseases or physiological abnormities by inhibiting inflammatory factors or angiogenesis. The water-solubility of the pharmaceutically acceptable strong acid salts of 3-(4-amino-1-oxo-1,3-dihydro-isoindol-2-yl)piperidine-2,6-dione is quite higher than that of 3-(4-amino-1-oxo-1,3-dihydro-isoindol-2-yl)piperidine-2,6-dione. The present invention also provides polymorphs of a pharmaceutically acceptable strong acid salt of 3-(4-amino-1-oxo-1,3-dihydro-isoindol-2-yl)piperidine-2,6-dione and a solvate thereof.

### FIELD

The present invention relates to a salt obtained by reacting 3-(4-amino-1-oxo-1,3-dihydro-isoindol-2-yl)piperidine-2,6-dione with various acids or a solvate of the salt, a polymorph of the salt or a solvate of the salt, and a process for preparing the same and use in the preparation of a medicament for treating diseases in which curative effects can be achieved by inhibiting inflammatory factors or angiogenesis.

### BACKGROUND

3-(4-amino-1-oxo-1,3-dihydro-isoindol-2-yl)piperidine-2,6-dione (II), in which Y represents H, has multiple pharmacological effects. Compound (II) can inhibit the in vivo release of inflammatory factors and also can inhibit angiogenesis. Therefore, it can be widely used for treating various diseases, which include but are not limited to inflammations, autoimmune abnormities, cancers and immunologic rejection syndromes (see U.S. Pat. Nos. 5,635,517 and 6,281,230, Chinese Patent No. ZL 200510013292.3).

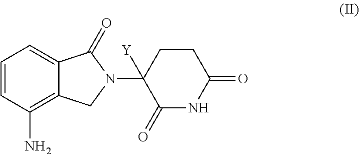

Tumor necrosis factor α (TNF α) is the main cytokine released by mononuclear phagocyte when it makes a response to an immune stimulation. The use of TNF α towards animal and human may result in inflammation, fever, cardiovascular function abnormity, hemorrhage, blood coagulation, and a series of acute reactions similar to acute infections and shock states. Excessive or uncontrolled TNF α produced in the animal or human body usually indicates the following diseases:

1. Endotoxemia and/or toxic shock syndrome [Tracey et al., Nature 330, 662-4 1987; Hinshaw et al., Circ Shock 30, 279-92 (1990)];

2. Cachexia [Dezube et al., Laucet, 335 (8690), 662 (1990)]; and

3. Adult respiration distress syndrome (ARDS) [Millar et al., Laucet 2 (8665), 712-714 (1989)].

TNF α further has an important effect on diseases regarding bone absorption including arthritis [Betolinni et al., Nature 319, 516-8 (1986)]. In vitro and in vivo tests demonstrate that TNF α can stimulate bone resorption by stimulating the creation and activation of osteoclast, and inhibit osteogenesis.

So far, the disease which has the most obvious relation with TNF α is hypercalcemia, in which the release of TNF α from tumor and host tissue is related with malignant tumor [Calci. Tissue Int. (US) 46 (Suppl.), S3-10 (1990)]. In the body of a patient receiving bone marrow transplantation, the immune reaction is closely related with the concentration of TNF α in the patient's serum [Holler et al., Blood, 75 (4), 1011-1016 (1990)].

The fatal, super-acute and neuropathic syndrome cerebral malaria is also related with the level of TNF α in blood. This disease is one of the most dangerous malarias. Upon its onset, the level of TNF α in serum is directly related with the patient's condition and it may generally happen in the body of a patient suffering from the onset of acute malaria [Grau et al., N. Engl. J. Med. 320 (24), 1586-91 (1989)].

TNF α also plays an important role in chronic pneumonia. The storage of silicon-containing particles in lung may result in silicosis. Silicosis is a progressive respiratory failure resulted from pulmonary fibrosis reaction. In the animal pathological model, the antibody of TNF α can completely prohibit the pulmonary fibrosis resulted from silicon dust in mice [Pignet et al., Nature, 344:245-7 (1990)]. Animal experiments also prove that the level of TNF α is abnormally high in serum of mice with pulmonary fibrosis resulted from silicon dust or asbestos dust [Bissonnette et al., Inflammation 13 (3), 329-339 (1989)]. Pathological studies disclose that the level of TNF α in lung tissue of a patient with sarcoidosis is also much higher than that of a normal people [Baughman et al., J. Lab. Cl in. Med. 115 (1), 36-42 (1990)], which indicates the inhibitor of TNF α has great significance on the treatment of chronic pulmonary disease and lung injury.

In patients suffering from reperfusion injury, the reason of inflammation may be also the abnormal level of TNF α in a patient's body. Furthermore, TNF α is considered to be responsible for tissue injury resulted from ischemia [Uadder et al., PNAS 87, 2643-6 (1990)].

Moreover, experiments indicate that TNF α can start the replication of retrovirus including HIV-1 [Duh et al., Proc. Nat. Acad. Sci., 86, 5974-8 (1989)]. The T-cells need to be activated before HIV enters into T-cells, and once the activated T-cells are infected by HIV virus, only these T-cells must continue to keep in the activated condition which can cause HIV virus genes to be successfully expressed and/or replicated. However, cytokines, in particular TNF α, play an important role in the process of HIV protein expression or the process of virus replication modulated by T-cell. Therefore, the inhibition of the TNF α formation may inhibit the replication of HIV virus in T-cell [Poll et al., Proc. Nat. Acad. Sci., 87, 782-5 (1990); Monto et al., Blood 79, 2670 (1990); Poll et al., AIDS Res. Human Retrovirus, 191-197 (1992)].

cAMP can modulate many cell functions, for example inflammation reaction including asthma and inflammation [Lowe and Chang, Drugs of the Future 17(9), 799-807, 1992]. Upon inflammation, the increase of the cAMP concentration of leucocyte inhibits the activation of leucocyte, and inflammation control factors including TNF α are then released, thereby inflammation in a patient is aggravated. Therefore, the inhibition of TNF α release can relieve inflammation diseases including asthma.

Recently, Yanyan Y U et al. found TNF α plays an important role in the progress of hepatic necrosis in a patient with viral hepatitis [Yanyan Yu et al., Chinese Journal of Internal Medicine 1996, 35: 28-31], which indicates that the inhibitor of TNF α has great significance in the treatment of chronic liver disease and liver injury.

Yingxu L I et al. found the level of synthesizing and secreting tumor necrosis factor by peripheral blood monocytes is increased significantly in a patient with chronic liver disease, and the secretion of other cell factors (e.g. I1-1β, I1-6 and I1-8) is also induced, which are jointly involved in the process of liver cell injury [Journal of Qiqihar Medical College, 22 (10): 1119-1120, 2001]. Their results are consistent with the conclusion of Yoshioka et al. [Hepatology, 1989, 10: 769-777] and Xin Wang et al. [Chinese Journal of Infectious Diseases, 1997, 15 (2): 85-88]. They further found thalidomide, a small molecular inhibitor of TNF α, can inhibit peripheral blood monocytes of a patient with hepatitis from secreting TNF α, which thereby builds the foundations for the inhibitors of TNF α to treat hepatitis, cirrhosis and liver cancer.

TNF α makes inflammatory cells aggregate and adhere, enhances the dilatation and permeability of microvessel, induces fever, increases circular neutrophils and changes the dynamic of blood by promoting the synthesis and release of inflammatory cell factor [Abboud H. E. Kidney Int. 1993; 43:252-267], inducing the expression of cell adhesion molecules [Egido J. et al Kidney Int. 1993; 43 (suppl 39): 59-64], and stimulating the synthesis and release of prostaglandin G₂ (PGE₂) and platelet activating factor (PAF) [Cammusi G. et al., Kidney Int., 43 (suppl 39): 32-36], thereby resulting in renal cell injury. Many studies demonstrate that TNF α plays an important role in the onset and aggravation of nephritis.

TNF α is involved in the regulation of immune function by activating macrophage, immuno-stimulating T lymphocyte proliferation, modulating B lymphocyte differentiation and enhancing the cytotoxicity of natural killer cells (NK).

Therefore, the decrease of the level of TNF α in a patient's body and/or the increase of the level of cAMP thereof is an effective way to treat many inflammatory diseases, infectious diseases, immune diseases or malignant tumor diseases, which include but are not limited to sepsis shock, endotoxic shock, hemodynamic shock, sepsis syndrome, post ischemic reperfusion injury, malaria, mycobacterial infection, meningitis, psoriasis, congestive heart failure, fibrotic disease, cachexia, transplantation immunological rejection, cancer, autoimmune disease, opportunistic infection in AIDS, rheumatoid arthritis (RA), hepatitis, nephritis, rheumatoid spondylitis and the like.

Some literatures have reported that on the lesion sites of Parkinson's disease, the content of inflammatory factors, in particular TNF, is extremely high. In the pathological model of Parkinson's disease, the inhibition of TNF release can reverse the progress of the disease, which indicates that the anti-TNF therapy can cure Parkinson's disease (McCoy, M. The Journal of Neuroscience, Sep. 13, 2006; vol 26).

Enbrel, a soluble TNF receptor, has better curative effects on arthritis and psoriasis, which shows that the inhibitors of TNF may treat arthritis and psoriasis.

Thalidomide has good curative effects on infantile meningitis in clinic, which indicates that the inhibitors of TNF may treat meningitis.

doctors observed that the level of TNF on the lesion sites of hepatitis is abnormally high, and further disclosed that TNF plays a central role in the progress of hepatic fibrosis and hepatic cirrhosis, which shows the inhibitors of TNF can be used to treat hepatitis and hepatic cirrhosis.

YU et al. observed the level of TNF is high in blood of a patient suffering from nephritis, which means the anti-TNF therapy may have curative effects on nephritis.

In view of the above, TNF antagonists may be used to treat diseases which can be treated by controlling the level of TNF, including but not limited to arthritis, hepatitis, gastritis, digestive ulcer, oral ulcer, nephritis, rhinitis, bronchitis, COPD (chronic obstructive pulmonary disease), pneumonia, pulmonary tuberculosis, myocarditis, pancreatitis, prostatitis, cervicitis, enteritis, Crohn's syndrome, nerve endings inflammation, myelitis, encephalitis, Parkinson's disease, psoriasis, lupus erythematosus, refractory dermatitis, leprosy, Parkinson's disease, progressive brain atrophy disease, Alzheimer's disease and hepatic cirrhosis. So the development of low-toxic and high-effective small molecular TNF α inhibitors has great social significance and economic value.

A compound of formula (II) can also inhibit angiogenesis and therefore can be widely used for treating various diseases, which include but are not limited to cancers. The cancers include but are not limited to bone marrow cancer, leukemia, liver cancer, brain tumor, prostatic cancer, gastric cancer, esophagus cancer, intestine cancer, laryngeal cancer, oral cancer, nose cancer, bone cancer, cervical cancer, lung cancer, breast cancer, renal cancer, lymphoma, ovarian cancer, pancreatic cancer, adrenal cancer, mesothelial cell cancer, melanoma and myelodysplastic syndrome.

Compound (II) has a poor water-solubility. U.S. Cellgene Corporation studied various hydrates and crystal forms of Compound (II) (see WO2005/023192). Through micronization technology, a capsule which is authorized by FDA for marketing is successfully developed.

For a particular population, oral formulation cannot achieve an effective bioavailability, in particular the water insoluble medicament such as a compound of formula (II), while injection of a medicament can achieve 100% bioavailability for all the patients.

From the analysis of the chemical structure characteristics and pKa of Compound (II), it can be concluded that Compound (II) is difficult to react with an acid to form a salt. However, it can easily react with a base to produce a metal salt such as a sodium salt, a potassium salt and a calcium salt. However, Compound (II) is unstable and easy to be hydrolyzed under basic conditions. Therefore, up to now a salt of Compound (II) has not been reported.

It is well-known that the preparation of a salt from a drug molecule in the form of free alkali not only can significantly increase the water-solubility and oral bioavailability of drug molecules, but also provides the possibility for forming the drugs into non-oral formulations such as intravenous formulation, intramuscular formulation, inhalant and drops. The preparation of a salt from a drug molecule in the form of free alkali also provides more choices for preparing a controlled release formulation. In addition, drug molecules in the form of salt can have advantages on the preparative technology of formulations such as better stability, machinability and the like.

The preparation of salt from drug molecules in the form of free alkali further provides the possibility for developing purification methods of bulk pharmaceuticals accepted by Drug Administration in various countries. Drugs in the form of salt can be recrystallized from safe solvents such as water, alcohols and ketones to obtain high-quality bulk pharmaceuticals. Therefore, the preparation of a salt from a drug molecule in the form of free alkali is generally the optimal choice of drug substance as raw materials.

In conclusion, it is necessary to successfully develop a salt of 3-(4-amino-1-oxo-1,3-dihydro-isoindol-2-yl)piperidine-2,6-dione.

### SUMMARY

When studying the relationship between the water-solubility of a compound represented by formula (II) and pH, the inventor found the water-solubility of a compound represented by formula (II) was increased under the acidic condition. It is disclosed that the amino groups in a compound represented by formula (I) can interact with hydrogen ion in XH. Subsequently, the inventor made a systematic study on the reaction of a compound represented by formula (II) with various inorganic acid and organic acid, and surprisingly found that a compound represented by formula (II) can react with strong acid to produce a salt. However, a compound represented by formula (II) cannot react with moderate and weak acids such as phosphoric acid, benzoic acid, succinic acid, oxalic acid, fumaric acid, maleic acid, acetic acid and the like to form a salt.

Accordingly, the present invention discloses a compound represented by formula (I), and a process for preparing the same and use thereof:

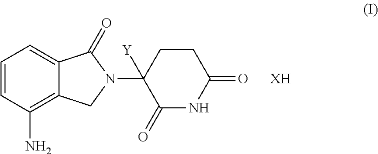

wherein Y represents H, CH₃ or F;

XH represents various pharmaceutically acceptable strong acids, which includes but is not limited to hydrochloric acid, hydrobromic acid, sulfuric acid, nitric acid or substituted sulfonic acid represented by formula (III).

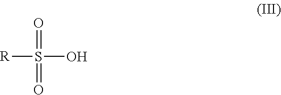

wherein R represents C₁₋₆ hydrocarbyl, aromatic ring or heterocyclic ring.

C₁₋₆ hydrocarbyl as used herein is a saturated branched hydrocarbyl, unsaturated branched hydrocarbyl, saturated straight hydrocarbyl, unsaturated straight hydrocarbyl, saturated cyclic hydrocarbyl or unsaturated cyclic hydrocarbyl, which may be optionally substituted with one or more substituent groups selected from the group consisting of F, Cl, Br, NO₂, OH, COOH, COOR¹, SO₃H, SO₂R¹, SOR¹, CN, CONR¹R², aromatic ring, aromatic heterocyclic ring, OR¹ and NR¹R².

Aromatic ring as used herein is a single aromatic ring or fused aromatic ring, which may be optionally substituted with one or more substituent groups selected from the group consisting of NO₂, F, Cl, Br, OH, COOH, NHC(O)R¹, NR¹R², NHR¹, NH₂, SR¹, OR¹, COOR¹ and SO₃H.

Heterocyclic ring as used herein includes a 4-, 5-, 6-, 7- or 8-membered saturated heterocyclic ring, unsaturated heterocyclic ring, aromatic heterocyclic ring or fused heterocyclic ring containing one or more heteroatoms such as nitrogen, oxygen and sulphur, which may be optionally substituted with one or more substituent groups selected from the group consisting of F, NO₂, Cl, Br, OH, COOH, NHC(O)R¹, NR¹R², NHR¹, NH₂, SR¹, OR¹ and COOR¹.

R¹ and R² used in the present invention independently represent a saturated or unsaturated branched or straight hydrocarbyl containing one to four carbon atoms, which may be optionally substituted with one or more substituent groups selected from the group consisting of F, Cl, Br, OH and COOH.

A compound which is suitable to form a salt with a compound of formula (II) is such a compound in which where R represents C₁₋₆ hydrocarbyl, R is selected from the group consisting of CH₃, CH₂CH₃, CH₂CH₂CH₃, CH₂CH₂CH₂CH₃, CH(CH₃)₂, CH₂CH(CH₃)₂, C(CH₃)₃, CF₃, CHF₂, CH₂F, CH₂CF₃, CH₂CHF₂, CH₂CH₂F, CH₂CH₂CF₃, CH₂CH₂CHF₂, CH₂CH₂CH₂F, cyclohexyl and cyclopentyl. A compound which is more suitable to form a salt with a compound of formula (II) is such a compound in which where R represents C₁₋₆ hydrocarbyl, R is selected from the group consisting of CH₃, CH₂CH₃, CH₂CH₂CH₃, CH(CH₃)₂, CH₂CH(CH₃)₂, CF₃, CHF₂, CH₂F, CH₂CF₃, CH₂CHF₂, CH₂CH₂F and CH₂CH₂CF₃. The compound which is most suitable to form a salt with a compound of formula (II) is such a compound in which where R represents C₁₋₆ hydrocarbyl, R is selected from the group consisting of CH₃, CH₂CH₃, CH₂CH₂CH₃, CF₃, CHF₂, CH₂F, CH₂CF₃ and CH₂CHF₂.

A compound represented by formula (III) which is suitable to form a salt with a compound of formula (II) is such a compound in which where R represents an aromatic ring, R is selected from the group consisting of phenyl, 1-naphthyl, 2-naphthyl, 5-sulphonatonaphthyl-1-yl, p-methylphenyl, o-methylphenyl, m-methylphenyl, p-hydroxyphenyl, o-hydroxyphenyl, m-hydroxyphenyl, trifluoromethylphenyl, p-carboxylphenyl, m-carboxylphenyl, o-carboxylphenyl, p-sulfophenyl, m-sulfophenyl, o-sulfophenyl, p-ethylphenyl, o-ethylphenyl, m-ethylphenyl, p-methoxyphenyl, m-methoxyphenyl, o-methoxyphenyl, 2,3-dimethylphenyl, 3,4-dimethylphenyl, o-nitrophenyl, m-nitrophenyl and p-nitrophenyl. A compound represented by formula (III) which is more suitable to form a salt with a compound of formula (II) is such a compound in which where R represents an aromatic ring, R is selected from the group consisting of phenyl, 1-naphthyl, 2-naphthyl, 5-sulphonatonaphthyl-1-yl, p-methylphenyl, o-methylphenyl, m-methylphenyl, p-hydroxyphenyl, o-hydroxyphenyl, m-hydroxyphenyl, trifluoromethylphenyl, p-ethylphenyl, o-ethylphenyl, m-ethylphenyl, p-methoxyphenyl, m-methoxyphenyl and o-methoxyphenyl. A compound represented by formula (III) which is most suitable to form salts with a compound of formula (II) is such a compound in which where R represents an aromatic ring, R is selected from the group consisting of phenyl, 1-naphthyl, 2-naphthyl, 5-sulphonatonaphthyl-1-yl, p-methylphenyl, o-methylphenyl, m-methylphenyl, p-hydroxyphenyl, o-hydroxyphenyl, m-hydroxyphenyl, trifluoromethylphenyl, p-ethylphenyl, o-ethylphenyl, m-ethylphenyl and p-methoxyphenyl.

A compound represented by formula (III) which is suitable to form a salt with a compound of formula (II) is such a compound in which where R represents heterocyclic ring, R is selected from the group consisting of furan ring, pyrrole ring, thiophene ring and pyridine ring.

“Strong acid” as used herein represents an organic or inorganic acid of which pKa is less than pKa1 of phosphoric acid.

The water-solubility of a compound represented by formula (I) is 4-5000 times higher than that of a compound represented by formula (II).

In a compound represented by formula (I), the molar ratio of Compound (II) to XH may be 1:0.1-5, more suitable molar ratio is 1:0.5-2, and the most suitable molar ratio is 1:1 or 2:1.

When a compound represented by formula (I) disclosed in the present invention is used, it may be solvent-free, and may also be solvated, in particular a hydrate or alcoholate.

When a compound represented by formula (I) disclosed in the present invention is used, it may be a single salt, and may also be a mixture of several salts.

When a compound represented by formula (I) disclosed in the present invention is used, it may be amorphous, and may also be in various crystal forms thereof or a mixture of these forms.

When a compound represented by formula (I) disclosed in the present invention is used, it may be S configuration, and may also be R configuration, or a mixture of R and S configurations.

A compound represented by formula (I) disclosed in the present invention may be prepared by the following processes:

Process A: A compound represented by formula (II) is dissolved in a suitable solvent, XH is added, the reaction temperature is controlled, the mixture is placed and the temperature is controlled.

Process B: XH is dissolved in a suitable solvent, a compound represented by formula (II) is added, the reaction temperature is controlled, the mixture is placed and the temperature is controlled.

Process C: A compound represented by formula (II) is dissolved in a suitable solvent, XH is dissolved in a suitable solvent and then added into a compound represented by formula (II), the reaction temperature is controlled, the mixture is placed and the temperature is controlled.

In the above preparation processes, the solvent may be any suitable solvent which includes but is not limited to water, alcohols, esters, ketones, ethers, amides, sulfones, sulfoxides or a mixture thereof.

In the above preparation processes, the suitable solvent of alcohols, esters, ketones, ethers, amides, sulfones or sulfoxides include but are not limited to methanol, ethanol, propanol, isopropanol, acetone, butanone, ethyl ether, isopropyl ether, THF (tetrahydrofuran), 1,4-dioxane, ethyl formate, ethyl acetate, propyl formate, isopropyl formate, methyl acetate, propyl acetate, isopropyl acetate, butyl formate, DMF (N,N-dimethylformamide), DMA (N,N-dimethylacetamide), DMSO (dimethyl sulfoxide) or a mixture thereof.

In the above preparation process, the molar ratio of a compound represented by formula (II) to XH may be 1:0.1-50, more suitable molar ratio is 1:0.2-10, and the most suitable molar ratio is 1:0.5-2. For example, where XH is a volatile acid, the addition amount of XH may be any amount which is more than 10% of the molar amount of a compound of formula (II).

In the above preparation processes, the reaction temperature may be −40° C. to 200° C., more suitable temperature is −20° C. to 100° C., and the most suitable temperature is 0° C. to 80° C.

The samples in examples were measured by mass spectrometry and H¹NMR. The measurement results demonstrate that the reaction is stable and there is no side reaction during the process of a reaction of a compound represented by formula (II) with XH to produce a salt.

The unit dose of a compound represented by formula (I) disclosed in the present invention is in the range of 0.1 mg to 250 mg, the optimized unit dose is 1 mg to 100 mg, and the optimal unit dose is 5 mg to 50 mg.

The most convenient unit dose of a compound represented by formula (I) disclosed in the present invention is the unit dose of a compound represented by formula (I) which is equivalent to 5 mg, 10 mg and 25 mg of a compound represented by formula (II), in which the most commonly used unit dose is the unit dose which is equivalent to 10 mg and 25 mg of a compound represented by formula (II).

In the present invention, the unit dose refers to a unit which can be administrated to a patient and can be readily operated and packaged, i.e., a single dosage.

When a compound represented by formula (I) is used as an active ingredient of a medicament, the indications are all the diseases which can be effectively relieved and treated by decreasing the level of TNF α in the body of a patient. The diseases include but are not limited to inflammatory diseases, infectious diseases, immune diseases or malignant tumor diseases. Specific diseases include but not limited to sepsis shock, endotoxic shock, hemodynamic shock, sepsis syndrome, post ischemic reperfusion injury, malaria, mycobacterial infection, meningitis, psoriasis, congestive heart failure, fibrotic disease, cachexia, transplantation immunological rejection, cancer, autoimmune disease, opportunistic infection in AIDS, erythema nodosum leprosum, lupus erythematosus, refractory lupus erythematosus, Behcet's Syndrome, regional ileitis, myelodysplastic syndrome, rheumatoid arthritis (RA), hepatitis, nephritis, rheumatoid spondylitis, multiple myeloma, thyroid tumor, renal cancer, prostatic cancer, lymphoma, leukemia and liver cancer.

A compound represented by formula (I) disclosed in the present invention may be formed into a tablet, a capsule, a powder injection, a solution formulation, a freeze-dried powder injection, an aerosol, a spray, a cream, a paste, eye drops, ear drops or an implant which can be administrated by oral administration, injection, inhalation, eye drops administration, ear drops administration, transdermal administration, rectal administration, vaginal administration and the like.

One advantage of a compound represented by formula (I) disclosed in the present invention is that it can prepared as an injection.

Another advantage of a compound represented by formula (I) disclosed in the present invention is that it can be prepared as a controlled release formulation.

A compound represented by formula (I) disclosed in the present invention may be used in combination with other suitable medicaments, which include but are not limited to oblimersen (Genasense®), remicade, docetaxel, celecoxib, melphalan, dexamethasone, steroid, gemcitabine, cisplatin, temozolomide, etoposide, cyclophosphamide, carboplatinum, procarbazine, carmustine, tamoxifen, topotecan, methotrexate, Arisa®, taxol, taxotere, fluorouracil, folinic acid, irinotecan, xeloda, CPT-11, interferon α, PEGylated interferon α, vinblastine, adriamycin, vincristine, sulindac and prednisone.

A compound represented by formula (I) disclosed in the present invention may also be used in combination with suitable extracts of traditional Chinese drugs.

The present invention provides use of a compound represented by formula (I) or a solvate thereof, in which XH represents various pharmaceutically acceptable strong acids. It is characterized in that the use is for the preparation of a medicament for treating diseases or physiological abnormalities in which curative effects can be achieved by inhibiting inflammatory factors.

The diseases or physiological abnormalities described in the present invention in curative effects which can be achieved by inhibiting inflammatory factors are arthritis, hepatitis, gastritis, digestive ulcer, oral ulcer, nephritis, rhinitis, bronchitis, COPD, pneumonia, pulmonary tuberculosis, myocarditis, pancreatitis, prostatitis, cervicitis, enteritis, Crohn's syndrome, nerve endings inflammation, myelitis, encephalitis, Parkinson's disease, psoriasis, lupus erythematosus, refractory dermatitis, leprosy, progressive brain atrophy disease, Alzheimer's disease and hepatic cirrhosis.

The present invention further provides use of a compound represented by formula (I) or a solvate thereof as inhibitors of angiogenesis, in which XH represents various pharmaceutically acceptable strong acids. It is characterized in that the use is for the preparation of a medicament for treating diseases in which curative effects can be achieved by inhibiting angiogenesis.

The diseases described in the present invention, in which curative effects can be achieved by inhibiting angiogenesis, are cancers, which include but are not limited to bone marrow cancer, leukemia, liver cancer, brain tumor, prostatic cancer, gastric cancer, esophagus cancer, intestine cancer, laryngeal cancer, oral cancer, nose cancer, bone cancer, cervical cancer, lung cancer, breast cancer, renal cancer, lymphoma, ovarian cancer, pancreatic cancer, adrenal cancer, mesothelial cell cancer, melanoma, myelodysplastic syndrome, bladder cancer, head and neck cancer, blood cancer, neuroblastoma, hemangiopericytoma and rectal cancer.

The present invention also discloses a polymorph of a compound represented by formula (I) or a solvate of a compound represented by formula (I):

Polymorph (IA) of a compound represented by formula (I) or a solvate of a compound represented by formula (I) where XH represents sulfuric acid,

Polymorph (IB) of a compound represented by formula (I) or a solvate of a compound represented by formula (I) where XH represents sulfuric acid,

Polymorph (IC) of a compound represented by formula (I) or a solvate of a compound represented by formula (I) where XH represents sulfuric acid,

Polymorph (IIA) of a compound represented by formula (I) or a solvate of a compound represented by formula (I) where XH represents nitric acid,

Polymorph (IIIA) of a compound represented by formula (I) or a solvate of a compound represented by formula (I) where XH represents benzene sulfonic acid,

Polymorph (IIIB) of a compound represented by formula (I) or a solvate of a compound represented by formula (I) where XH represents benzene sulfonic acid,

Polymorph (IVA) of a compound represented by formula (I) or a solvate of a compound represented by formula (I) where XH represents p-toluene sulfonic acid,

Polymorph (IVB) of a compound represented by formula (I) or a solvate of a compound represented by formula (I) where XH represents p-toluene sulfonic acid,

Polymorph (VA) of a compound represented by formula (I) or a solvate of a compound represented by formula (I) where XH represents hydrobromic acid,

Polymorph (VIA) of a compound represented by formula (I) or a solvate of a compound represented by formula (I) where XH represents methylsulfonic acid,

Polymorph (VIB) of a compound represented by formula (I) or a solvate of a compound represented by formula (I) where XH represents methylsulfonic acid,

Polymorph (VIIA) of a compound represented by formula (I) or a solvate of a compound represented by formula (I) where XH represents hydrochloric acid,

### Measuring Conditions of X-Ray Powder Diffraction Spectra:

Sample weight: about 100 mg

### Target: Cu

Filter: homochromy
Voltage/Current: 40 kV/100 mA

### Slit: SS/DS 1°, RS 0.3 mm

Scanning speed: 8°/min

### Measuring Conditions of Infrared Absorption:

Resolution: 4 cm⁻¹
Scanning number: 20
Spectral range: 4000-400 cm⁻¹

### Measuring Conditions of Thermal Analysis and Differential Thermal Analysis (TG-DTA):

Sample weight: about 6 mg
Reference substance: Al₂O₃
Heating rate: 10° C./min
Sample injection: 0.7 seconds
Upper limit: 250° C.
Lower limit: room temperature

### (1) Polymorph (IA)

Parameters in X-ray powder diffraction spectra:

| Diffraction Angles (2θ, °) | Strength (I/I₀) |
|---|---|
| 16.52 | 74.7 |
| 17.04 | 16.4 |
| 18.763 | 10.9 |
| 19.46 | 100 |
| 20.422 | 25.6 |
| 20.817 | 16.8 |
| 21.940 | 32.4 |
| 22.321 | 63.2 |
| 25.403 | 14.8 |
| 26.098 | 95.7 |
| 26.78 | 37.8 |
| 29.084 | 16.5 |
| 34.715 | 11.1 |

### (2) Polymorph (IB)

Parameters in X-ray powder diffraction spectra:

| Diffraction Angles (2θ, °) | Strength (I/I₀) |
|---|---|
| 5.580 | 53.6 |
| 10.437 | 17.3 |
| 12.820 | 50.0 |
| 16.759 | 79.6 |
| 17.139 | 19.6 |
| 18.860 | 100 |
| 19.241 | 38.2 |
| 20.641 | 54.6 |
| 21.099 | 37.3 |
| 21.700 | 22.5 |
| 22.759 | 21.0 |
| 24.158 | 14.4 |
| 25.118 | 15.8 |
| 26.739 | 67.9 |
| 27.359 | 40.1 |
| 30.020 | 13.6 |

The wavelength of infrared absorption spectra of Polymorph (IB) in potassium bromide (nm):

| | | | | | | | | |
|---|---|---|---|---|---|---|---|---|
| 453.2 | 526.48 | 653.76 | 692.33 | 767.54 | 806.11 | 844.68 | 950.75 | 1033.7 |
| 1076.1 | 1286.3 | 1315.2 | 1348 | 1400.1 | 1436.7 | 1504.2 | 1571.7 | 1618 |
| 1889.9 | 1951.6 | 2159.9 | 2698 | 3037.4 | 3172.4 | 3328.6 | 3810.7 | 3938 |

### (3) Polymorph (IC)

Parameters in X-ray powder diffraction spectra:

| Diffraction Angles (2θ, °) | Strength (I/I₀) |
|---|---|
| 14.243 | 46.5 |
| 16.392 | 22.2 |
| 16.919 | 99.3 |
| 17.183 | 40.6 |
| 18.780 | 83.7 |
| 20.376 | 22.4 |
| 21.438 | 67.2 |
| 23.380 | 34.9 |
| 24.060 | 25.5 |
| 25.645 | 100 |
| 26.584 | 47.4 |
| 27.339 | 25.9 |
| 28.076 | 45.3 |
| 28.282 | 22.6 |
| 28.833 | 20.8 |
| 30.261 | 32.8 |
| 32.486 | 25.7 |

### (4) Polymorph (IIA)

Parameters in X-ray powder diffraction spectra:

| Diffraction Angles (2θ, °) | Strength (I/I₀) |
|---|---|
| 5.861 | 51.6 |
| 7.958 | 19.5 |
| 11.720 | 75.4 |
| 13.221 | 17.0 |
| 14.418 | 50.4 |
| 15.518 | 18.3 |
| 16.199 | 34.5 |
| 16.701 | 81.1 |
| 17.701 | 30.1 |
| 18.680 | 28.6 |
| 20.618 | 100 |
| 23.860 | 69.2 |
| 25.240 | 27.5 |
| 26.600 | 81.8 |
| 27.320 | 96.7 |
| 29.361 | 27.3 |

### (5) Polymorph (IIIA)

Parameters in X-ray powder diffraction spectra:

| Diffraction Angles (2θ, °) | Strength (I/I₀) |
|---|---|
| 4.799 | 100 |
| 9.681 | 13.6 |
| 13.818 | 23.4 |
| 15.418 | 36.6 |
| 17.658 | 46.3 |
| 18.602 | 20.9 |
| 20.799 | 30.7 |
| 22.201 | 23.7 |
| 24.041 | 37.2 |
| 24.559 | 29.6 |
| 26.580 | 16.6 |

### (6) Polymorph (IIIB)

Parameters in X-ray powder diffraction spectra:

| Diffraction Angles (2θ, °) | Strength (I/I₀) |
|---|---|
| 5.440 | 46.0 |
| 10.981 | 17.2 |
| 13.080 | 26.9 |
| 14.761 | 18.6 |
| 15.800 | 24.1 |
| 17.041 | 85.2 |
| 17.420 | 21.6 |
| 19.119 | 38.3 |
| 19.600 | 34.9 |
| 20.040 | 100 |
| 24.981 | 74.2 |
| 26.322 | 35.1 |
| 28.782 | 22.7 |

The wavelength of infrared absorption spectra of Polymorph (IIIB) in potassium bromide (nm):

| | | | | | | | | |
|---|---|---|---|---|---|---|---|---|
| 403.06 | 445.48 | 495.62 | 528.41 | 582.41 | 653.76 | 711.62 | 777.18 | 846.61 |
| 900.61 | 946.89 | 1062.6 | 1157.1 | 1215 | 1280.5 | 1313.3 | 1346.1 | 1394.3 |
| 1434.8 | 1477.2 | 1506.2 | 1573.7 | 1841.7 | 1878.4 | 2179.2 | 2723 | 3039.3 |
| 3135.7 | 3301.6 | 3708.5 | 3741.3 | 3799.1 | 3851.2 | | | |

### (7) Polymorph (IVA)

Parameters in X-ray powder diffraction spectra:

| Diffraction Angles (2θ, °) | Strength (I/I₀) |
|---|---|
| 4.640 | 100 |
| 13.580 | 32.3 |
| 14.360 | 17.1 |
| 15.200 | 21.1 |
| 17.399 | 59.5 |
| 19.339 | 15.7 |
| 19.938 | 18.8 |
| 20.560 | 24.3 |
| 24.181 | 68.0 |
| 27.583 | 15.3 |

The wavelength of infrared absorption spectra of Polymorph (IVA) in potassium bromide (nm)

| | | | | | | | | |
|---|---|---|---|---|---|---|---|---|
| 441.63 | 526.48 | 649.9 | 721.26 | 771.4 | 896.75 | 948.82 | 1020.2 | 1060.7 |
| 1137.8 | 1218.8 | 1282.5 | 1313.3 | 1394.3 | 1432.9 | 1475.3 | 1508.1 | 1619.9 |
| 1878.4 | 1940.1 | 1994.1 | 2144.5 | 2715.3 | 3012.3 | 3162.7 | 3299.7 | 3812.6 |
| 3939.9 | | | | | | | | |

### (8) Polymorph (IVB)

Parameters in X-ray powder diffraction spectra:

| Diffraction Angles (2θ, °) | Strength (I/I₀) |
|---|---|
| 12.438 | 20.80 |
| 15.522 | 16.2 |
| 18.900 | 83.89 |
| 19.799 | 100 |
| 21.039 | 17.7 |
| 26.662 | 28.6 |

### (9) Polymorph (VA)

Parameters in X-ray powder diffraction spectra:

| Diffraction Angles (2θ, °) | Strength (I/I₀) |
|---|---|
| 10.040 | 27.2 |
| 11.963 | 29.1 |
| 15.118 | 17.7 |
| 16.879 | 50.9 |
| 20.479 | 41.6 |
| 20.896 | 18.7 |
| 21.481 | 100 |
| 22.760 | 23.1 |
| 23.520 | 19.8 |
| 24.199 | 52.1 |
| 24.639 | 91.5 |
| 25.920 | 70.1 |
| 26.839 | 42.2 |
| 28.140 | 30.5 |
| 30.040 | 20.7 |
| 31.120 | 36.6 |
| 33.539 | 24.6 |
| 35.081 | 19.4 |

### (10) Polymorph (VIA)

Parameters in X-ray powder diffraction spectra:

| Diffraction Angles (2θ, °) | Strength (I/I₀) |
|---|---|
| 7.459 | 10.5 |
| 11.519 | 31.1 |
| 14.798 | 34.1 |
| 14.999 | 18.3 |
| 15.719 | 19.5 |
| 17.278 | 38.9 |
| 19.179 | 44.5 |
| 19.500 | 39.1 |
| 21.901 | 100 |
| 23.721 | 29.1 |
| 26.481 | 65.7 |
| 27.721 | 18.2 |

### (11) Polymorph (VIB)

Parameters in X-ray powder diffraction spectra:

| Diffraction Angles (2θ, °) | Strength (I/I₀) |
|---|---|
| 9.941 | 32.5 |
| 10.937 | 47.1 |
| 15.341 | 54.0 |
| 16.501 | 69.9 |
| 18.360 | 33.0 |
| 18.919 | 85.8 |
| 19.458 | 63.6 |
| 20.020 | 22.8 |
| 21.040 | 82.8 |
| 22.080 | 21.3 |
| 23.641 | 36.3 |
| 24.161 | 57.5 |
| 25.539 | 100 |
| 28.200 | 22.2 |
| 30.500 | 15.3 |
| 34.601 | 15.6 |

### (12) Polymorph (VIIA)

Parameters in X-ray powder diffraction spectra:

| Diffraction Angles (2θ, °) | Strength (I/I₀) |
|---|---|
| 10.196 | 15.9 |
| 12.018 | 55.2 |
| 13.162 | 41.9 |
| 15.279 | 15.7 |
| 17.020 | 40.8 |
| 19.058 | 25.4 |
| 20.460 | 40.7 |
| 21.580 | 49.4 |
| 22.478 | 17.6 |
| 22.980 | 19.1 |
| 24.241 | 52.8 |
| 24.642 | 100 |
| 25.420 | 32.2 |
| 26.042 | 48.0 |
| 26.581 | 30.0 |
| 26.820 | 36.7 |
| 28.302 | 23.2 |
| 30.359 | 16.7 |
| 31.081 | 25.5 |
| 35.301 | 22.4 |

The wavelength of infrared absorption spectra of Polymorph (IVA) in potassium bromide (nm):

| | | | | | | | | |
|---|---|---|---|---|---|---|---|---|
| 431.64 | 497.22 | 526.16 | 564.73 | 651.53 | 697.82 | 771.12 | 884.92 | 948.57 |
| 1029.6 | 1068.2 | 1218.6 | 1278.4 | 1311.2 | 1349.8 | 1390.3 | 1438.5 | 1471.3 |
| 1500.2 | 1540.7 | 1621.7 | 1849.3 | 1870.6 | 1920.7 | 2100.1 | 2119.4 | 2138.7 |
| 2171.5 | 2221.6 | 2320 | 2348.9 | 2661.4 | 3010.5 | 3029.8 | 3251.6 | 3679.8 |
| 3710.7 | 3749.3 | 3809 | 3859.2 | 3909.3 | 3930.6 | 3971.1 | | |

The present invention also provides a process for preparing a polymorph of a compound represented by formula (I) or a solvate of a compound represented by formula (I):

Process A: A compound represented by formula (II) is dissolved in a suitable solvent, XH is added, and the resultant mixture is stirred and filtered.

Process B: XH is dissolved in a suitable solvent, a compound represented by formula (II) is added, and the resultant mixture is stirred and filtered.

Process C: A compound represented by formula (II) is dissolved in a suitable solvent; XH is dissolved in a suitable solvent and then added to the solution comprising the compound represented by formula (II), and the resultant mixture is stirred and filtered.

In the above preparation processes, the solvent may be any suitable solvent, which includes but is not limited to water, alcohols, esters, ketones, ethers, amides, sulfones, sulfoxides or a mixture thereof.

In the above preparation processes, the suitable solvents of alcohols, esters, ketones, ethers, amides, sulfones or sulfoxides include but are not limited to methanol, ethanol, propanol, isopropanol, acetone, butanone, ethyl ether, isopropyl ether, THF (tetrahydrofuran), 1,4-dioxane, ethyl formate, ethyl acetate, propyl formate, isopropyl formate, methyl acetate, propyl acetate, isopropyl acetate, butyl formate, DMF (N,N-dimethylformamide), DMA (N,N-dimethyl-acetamide), DMSO (dimethyl sulfoxide) or a mixture thereof.

In the above preparation processes, the molar ratio of the compound represented by formula (II) to XH may be 1:0.1-50, more suitable molar ratio is 1:0.2-10, and the most suitable molar ratio is 1:0.5-2. For example, where XH is a volatile acid, the addition amount of XH may be any amount which is more than 10% of the molar amount of the compound of formula (II).

In the above preparation processes, the reaction temperature may be −40° C. to 200° C., more suitable temperature is −20° C. to 100° C., and the most suitable temperature is 0° C. to 80° C.

The unit dose of a polymorph of a compound represented by formula (I) or a solvates of a compound represented by formula (I) disclosed in the present invention is in the range of 0.1 mg to 250 mg, the optimized unit dose is 1 mg to 100 mg, and the optimal unit dose is 5 mg to 50 mg.

The most convenient unit dose of a polymorph of a compound represented by formula (I) or a solvate of a compound represented by formula (I) disclosed in the present invention is a unit dose of a compound represented by formula (I) which is equivalent to 5 mg, 10 mg and 25 mg of a compound represented by formula (II). The most commonly used unit dose is a unit dose which is equivalent to 10 mg and 25 mg of a compound represented by formula (II).

In the present invention, the unit dose refers to a unit which can be administrated to a patient and can be readily operated and packaged, i.e., a single dosage.

When a polymorph of a compound represented by formula (I) or a solvate of a compound represented by formula (I) is used as an active ingredient of a medicament, the indications are all the diseases which can be effectively relieved and treated by decreasing the level of TNF α in the body of a patient. The diseases include but are not limited to inflammatory diseases, infectious diseases, immune diseases or malignant tumor diseases. Specific diseases include but are not limited to sepsis shock, endotoxic shock, hemodynamic shock, sepsis syndrome, post ischemic reperfusion injury, malaria, mycobacterial infection, meningitis, psoriasis, congestive heart failure, fibrotic disease, cachexia, transplantation immunological rejection, cancer, autoimmune disease, opportunistic infection in AIDS, erythema nodosum leprosum, lupus erythematosus, refractory lupus erythematosus, Behcet's Syndrome, regional ileitis, myelodysplastic syndrome, rheumatoid arthritis (RA), hepatitis, nephritis, rheumatoid spondylitis, multiple myeloma, thyroid tumor, renal cancer, prostatic cancer, lymphoma, leukemia and liver cancer.

A polymorph of a compound represented by formula (I) or a solvate of a compound represented by formula (I) disclosed in the present invention may be formed into a formulation which can be administrated by oral administration, injection, inhalation, eye drops administration, ear drops administration, transdermal administration, rectal administration, vaginal administration and the like. The formulation includes but is not limited to an injection, a powder injection, a freeze-dried powder injection, a tablet, a capsule, a dropping pill, a spray, eye drops, ear drops, a paste, a cream, an implant, a controlled release formulation or a solution formulation.

A polymorph of a compound represented by formula (I) or a solvate of a compound represented by formula (I) disclosed in the present invention may be used in combination with other suitable medicaments, which include but are not limited to at least one of oblimersen (Genasense®), remicade, docetaxel, celecoxib, melphalan, dexamethasone, steroid, gemcitabine, cisplatin, temozolomide, etoposide, cyclophosphamide, carboplatinum, procarbazine, carmustine, tamoxifen, topotecan, methotrexate, Arisa®, taxol, taxotere, fluorouracil, folinic acid, irinotecan, xeloda, CPT-11, interferon α, PEGylated interferon α, vinblastine, adriamycin, vincristine, sulindac or prednisone.

A polymorph of a compound represented by formula (I) or a solvate of a compound represented by formula (I) disclosed in the present invention may be used in combination with suitable extracts of traditional Chinese drugs.

The present invention provides use of a polymorph of a compound represented by formula (I) or a solvate of a compound represented by formula (I), in which XH represents various pharmaceutically acceptable strong acids. It is characterized in that the use is for the preparation of a medicament for treating diseases or physiological abnormalities in which curative effects can be achieved by inhibiting inflammatory factors.

The diseases or physiological abnormalities described in the present invention, in which curative effects can be achieved by inhibiting inflammatory factors, include but are not limited to arthritis, hepatitis, gastritis, digestive ulcer, oral ulcer, nephritis, rhinitis, bronchitis, COPD, pneumonia, pulmonary tuberculosis, myocarditis, pancreatitis, prostatitis, cervicitis, enteritis, Crohn's syndrome, nerve endings inflammation, myelitis, encephalitis, parkinson disease, psoriasis, lupus erythematosus, refractory dermatitis, leprosy, Parkinson's disease, progressive brain atrophy disease, Alzheimer's disease or hepatic cirrhosis.

The present invention further provides use of a polymorph of a compound represented by formula (I) or a solvate of a compound represented by formula (I) as inhibitors of angiogenesis, in which XH represents various pharmaceutically acceptable strong acids. It is characterized in that the use is for the preparation of a medicament for treating diseases in which curative effects can be achieved by inhibiting angiogenesis.

The diseases described in the present invention, in which curative effects can be achieved by inhibiting angiogenesis, are cancers, which include but are not limited to bone marrow cancer, leukemia, liver cancer, brain tumor, prostatic cancer, gastric cancer, esophagus cancer, intestine cancer, laryngeal cancer, oral cancer, nose cancer, bone cancer, cervical cancer, lung cancer, breast cancer, renal cancer, lymphoma, ovarian cancer, pancreatic cancer, adrenal cancer, mesothelial cell cancer, melanoma, myelodysplastic syndrome, bladder cancer, head and neck cancer, blood cancer, neuroblastoma, hemangiopericytoma or rectal cancer.

### DESCRIPTION OF THE DRAWINGS

FIG. 1 shows the X-ray powder diffraction spectra of polymorph (IA) of a compound represented by formula (I) or a solvate of a compound represented by formula (I) where XH represents sulfuric acid.

FIG. 2 shows the X-ray powder diffraction spectra of polymorph (IB) of a compound represented by formula (I) or a solvate of a compound represented by formula (I) where XH represents sulfuric acid.

FIG. 3 shows the X-ray powder diffraction spectra of polymorph (IC) of a compound represented by formula (I) or a solvate of a compound represented by formula (I) where XH represents sulfuric acid.

FIG. 4 shows the X-ray powder diffraction spectra of polymorph (IIA) of a compound represented by formula (I) or a solvate of a compound represented by formula (I) where XH represents nitric acid.

FIG. 5 shows the X-ray powder diffraction spectra of polymorph (IIIA) of a compound represented by formula (I) or a solvate of a compound represented by formula (I) where XH represents benzene sulfonic acid.

FIG. 6 shows the X-ray powder diffraction spectra of polymorph (IIIB) of a compound represented by formula (I) or a solvate of a compound represented by formula (I) where XH represents benzene sulfonic acid.

FIG. 7 shows the X-ray powder diffraction spectra of polymorph (IVA) of a compound represented by formula (I) or a solvate of a compound represented by formula (I) where XH represents p-toluene sulfonic acid.

FIG. 8 shows the X-ray powder diffraction spectra of polymorph (IVB) of a compound represented by formula (I) or a solvate of a compound represented by formula (I) where XH represents p-toluene sulfonic acid.

FIG. 9 shows the X-ray powder diffraction spectra of polymorph (VA) of a compound represented by formula (I) or a solvate of a compound represented by formula (I) where XH represents hydrobromic acid.

FIG. 10 shows the X-ray powder diffraction spectra of polymorph (VIA) of a compound represented by formula (I) or a solvate of a compound represented by formula (I) where XH represents methylsulfonic acid.

FIG. 11 shows the X-ray powder diffraction spectra of polymorph (VIB) of a compound represented by formula (I) or a solvate of a compound represented by formula (I) where XH represents methylsulfonic acid.

FIG. 12 shows the X-ray powder diffraction spectra of polymorph (VIIA) of a compound represented by formula (I) or a solvate of a compound represented by formula (I) where XH represents hydrochloric acid.

FIG. 13 shows the infrared absorption spectra of polymorph (IB) of a compound represented by formula (I) or a solvate of a compound represented by formula (I) where XH represents sulfuric acid.

FIG. 14 shows the infrared absorption spectra of polymorph (IIIB) of a compound represented by formula (I) or a solvate of a compound represented by formula (I) where XH represents benzene sulfonic acid.

FIG. 15 shows the infrared absorption spectra of polymorph (IVA) of a compound represented by formula (I) or a solvate of a compound represented by formula (I) where XH represents p-toluene sulfonic acid.

FIG. 16 shows the infrared absorption spectra of polymorph (VIIA) of a compound represented by formula (I) or a solvate of a compound represented by formula (I) where XH represents hydrochloric acid.

FIG. 17 shows the standard line of the solubility determination.

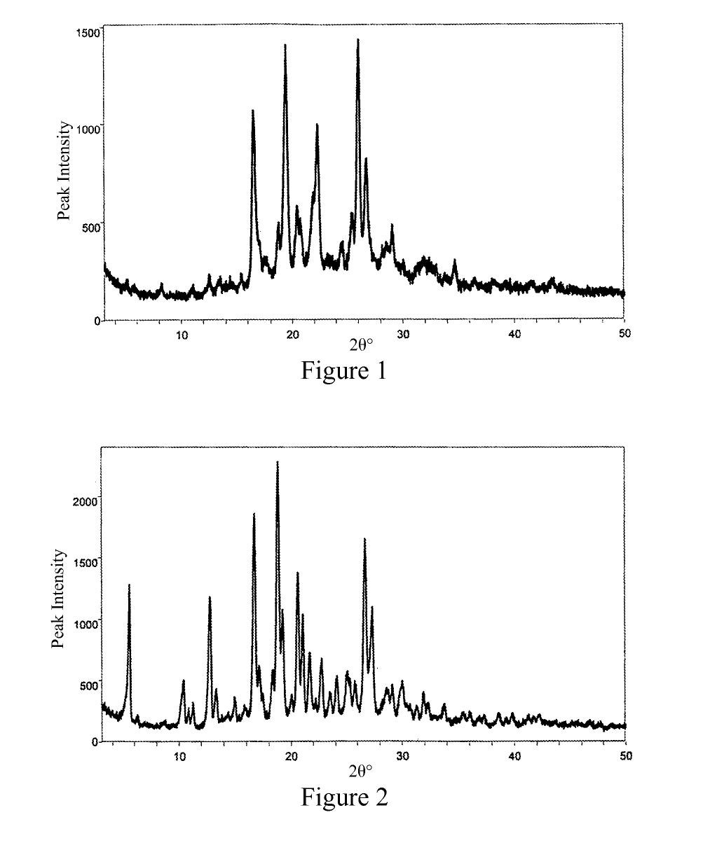

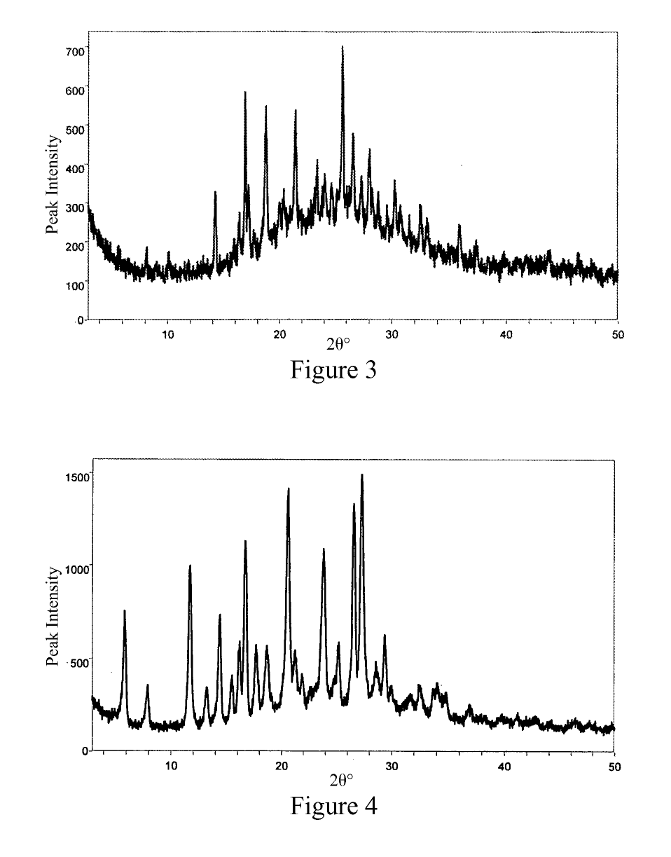

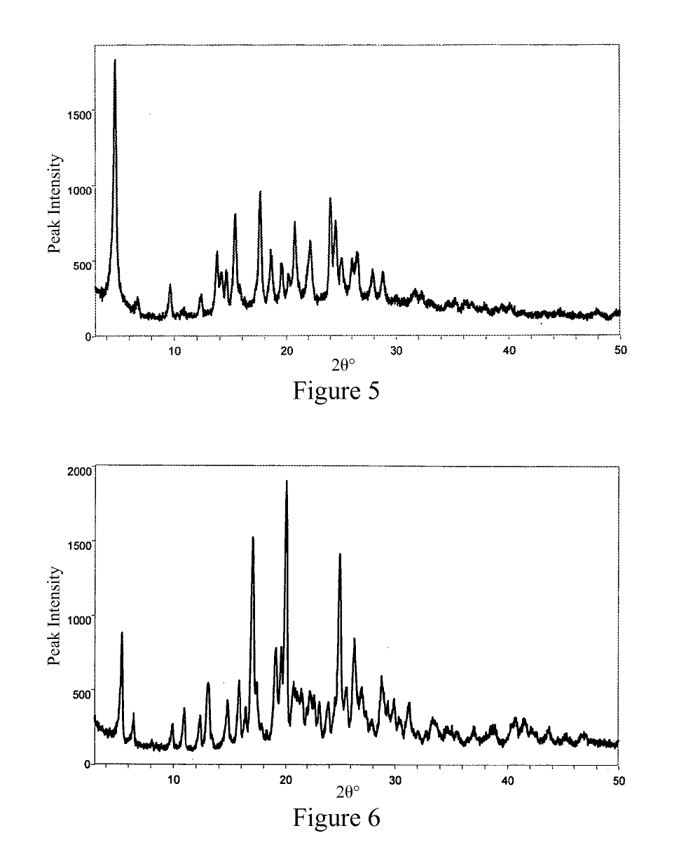

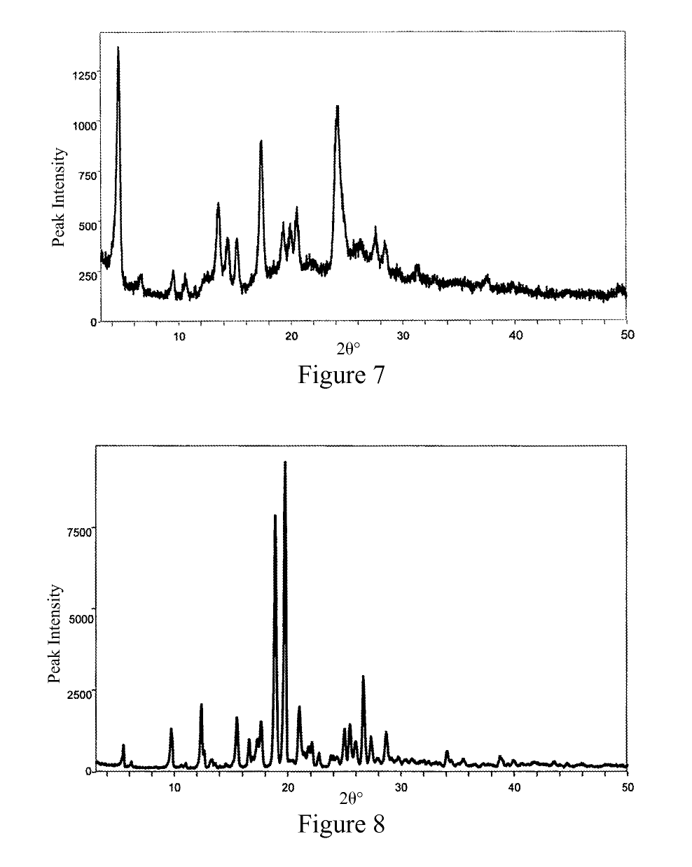

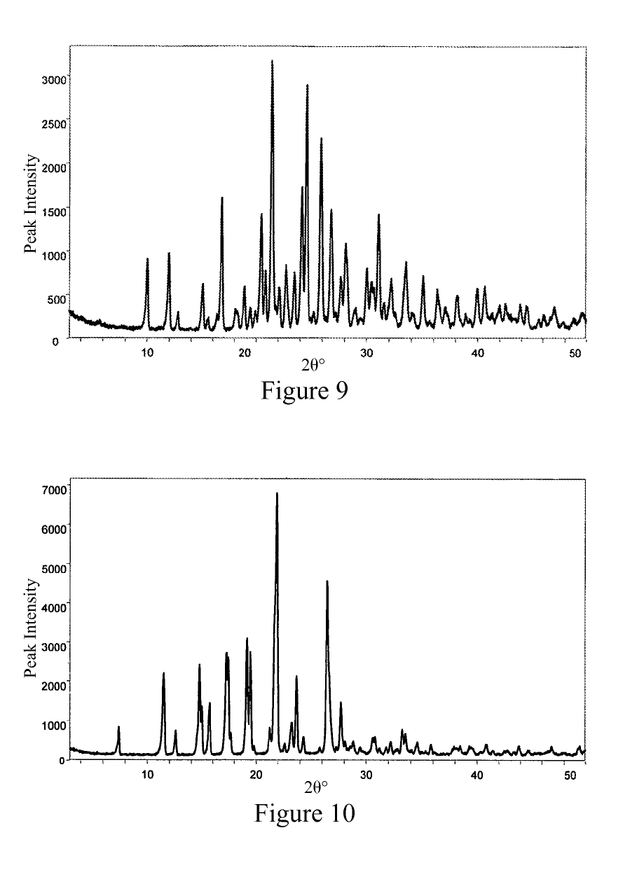

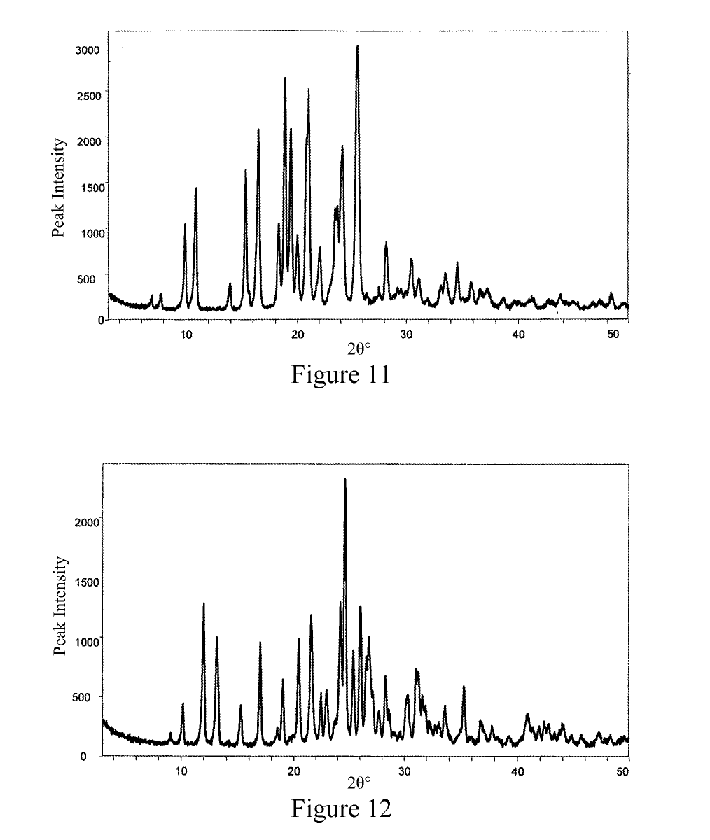

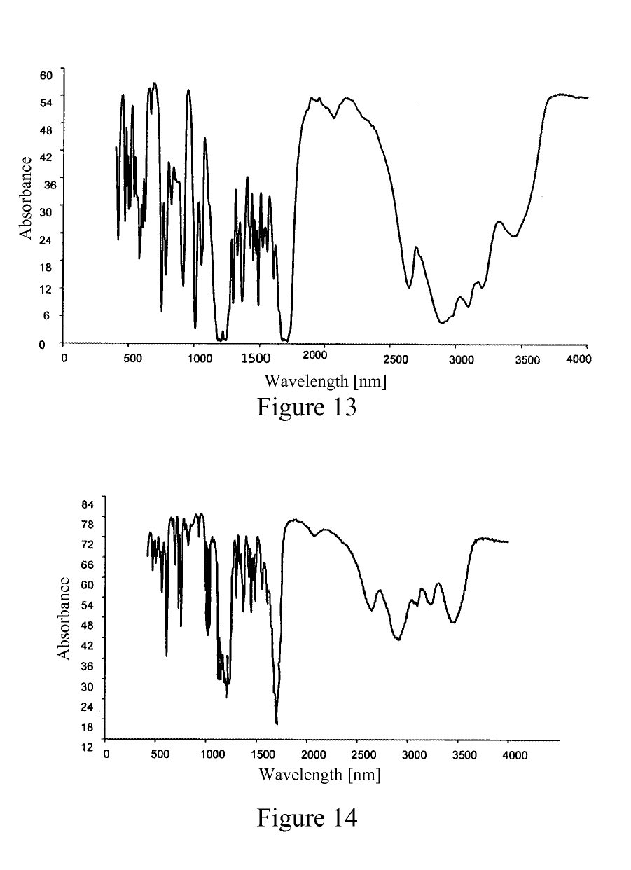

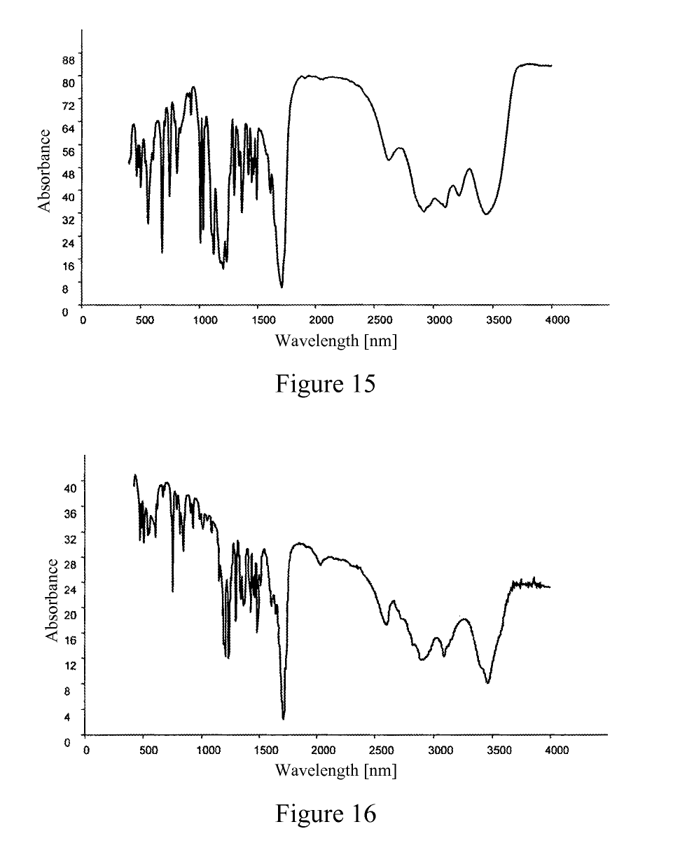

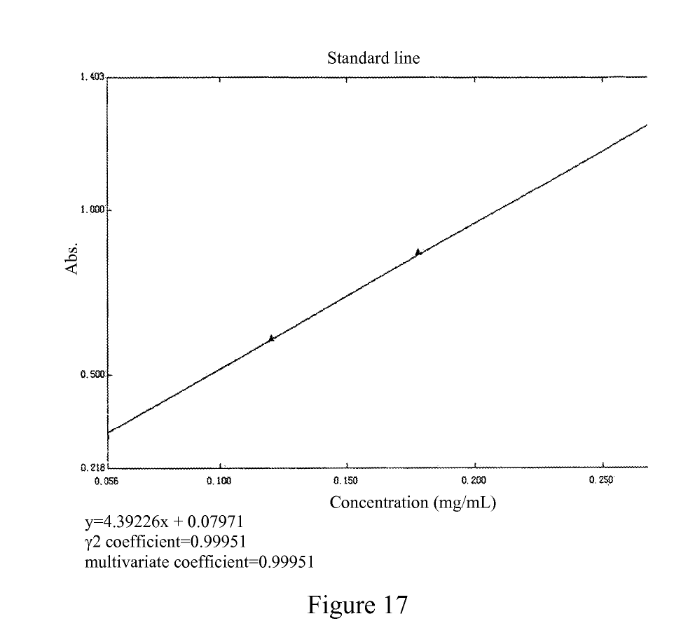

### SPECIFIC EMBODIMENTS

Abbreviations: mg: milligram, kg: kilogram, mL: milliliter.

### Preparation of Strong Organic Acid Salts of Compound (II)

### Examples 1-6

To ethanol were added a compound represented by formula (II), in which Y represents H, (518 mg) and an equal mole of an organic acid, The resultant mixture was heated and stirred. A small amount of water was added. The mixture was stirred for further 30 mins, and then was stirred at room temperature. After a solid precipitated, the mixture was stirred for further 4 hours and filtered by pump. The filter cake was dried for 10 hours in vacuo. The solubility of the resulting salts (1:1) in water was shown in Table 1.

TABLE 1
Solubility of strong organic acid salt of Compound
(II), in which Y represents H in water.

| | | Solubility |
|---|---|---|
| Example | Organic acid | (mg/mL, 15° C.) |
| Example 1 | benzene sulfonic acid | 3.76 |
| Example 2 | methylsulfonic acid | 1.85 |
| Example 3 | p-toluene sulfonic acid | 7.03 |
| Example 4 | 1-naphthalenesulfonic acid | 5.60 |
| Example 5 | 2-naphthalenesulfonic acid | 6.20 |
| Example 6 | 1,5-naphthalene disulfonic acid | >500 |
| Compound (II), | Free base•H₂O | 0.29 |
| in which Y | | |
| represents H | | |

### Example 7

To ethanol was added a compound represented by formula (II), in which Y represents H, (518 mg). 1 mL of sulfuric acid (98%) was slowly added under stirring to the resultant mixture at room temperature and stirred until the system became clear. After stirring overnight, a solid was obtained and filtered with pump. The filter cake was dried for 10 hours in vacuo. A bisulfate of Compound (II) (II:sulfuric acid=1:1) with four crystal water was obtained. Elemental analysis: C₁₃H₁₃N₃O₃.H₂SO₄.4H₂O, Calculated values: C, 36.36%, H, 5.36%, N, 9.79%. Measured values: C, 35.79%, H, 4.59%, N, 9.68%. Melting point: decomposition at 277.0-278.8° C., Water-solubility: >1000 mg/ml (15° C.).

### Example 8

To ethanol was added a compound represented by formula (II), in which Y represents CH₃ (546 mg). 1 mL of sulfuric acid (98%) was slowly added under stirring to the resultant mixture at room temperature and stirred until the system became clear. After stirring overnight, a solid was obtained and filtered with pump. The filter cake was dried for 10 hours in vacuo. A bisulfate of Compound (II) (II:sulfonic acid=1:1) with one crystal water was obtained.

### Example 9

To ethanol was added a compound represented by formula (II), in which Y represents F, (547 mg). 1 mL of sulfuric acid (98%) was slowly added under stirring to the resultant mixture at room temperature and stirred until the system became clear. After stirring overnight, a solid was obtained and filtered with pump. The filter cake was dried for 10 hours in vacuo. A bisulfate of Compound (II), in which Y represents F, (II:sulfuric acid=1:1) with one crystal water was obtained.

### Example 10

To 30 mL of anhydrous ethanol was added a compound represented by formula (II), in which Y represents H, (518 mg)., 1 mL of concentrated nitric acid (65%) was added to the anhydrous ethanol solution of Compound (II) under stirring at room temperature. The system was clear. After stirring for 2 hours, a solid precipitated and the solid was filtered with pump. The filter cake was dried for 10 hours in vacuo. A nitrate of Compound (II) (1:1) (containing two crystal water) was obtained. Elemental analysis: C₁₃H₁₃N₃O₃.HNO₃.2H₂O, Calculated values: C, 43.58%; H, 5.03%; N, 15.46%. Measured values: C, 43.83%; H, 4.87%; N, 16.98%. Melting point: 225.5° C., Water-solubility: 3.68 mg/ml (15° C.).

### Example 11

To 30 mL of anhydrous ethanol was added a compound represented by formula (II), in which Y represents H, (518 mg). 1 mL of concentrated hydrochloric acid (38%) was added. The mixture was heated and stirred. A small amount of water was added to make the system clear. At room temperature, the mixture was further stirred. A solid precipitated and the solid was filtered with pump. The filter cake was dried for 10 hours in vacuo. A hydrochlorate of Compound (II) (1:1) (containing one crystal water) was obtained. Elemental analysis: C₁₃H₁₃N₃O₃.HCl.1H₂O, Calculated values: C, 43.58%; H, 5.03%; N, 15.46%. Measured values: C, 43.83%; H, 4.87%; N, 16.98%. Melting point: 238.5-238.7° C., Water-solubility: 1.23 mg/ml (15° C.).

### Example 12

To 30 mL of anhydrous ethanol was added a compound represented by formula (II), in which Y represents H, (518 mg). 1 mL of concentrated phosphoric acid (85%) was added, and then the mixture was put into an oil bath and heated until reflux was happened. A small amount of water was added to make the system clear. The mixture was refluxed for 30 min, and then stirred at room temperature. After a solid precipitated, the mixture was stirred for further 4 hours, and then filtered with pump. The filter cake was dried for 10 hours in vacuo. The raw material (II), in which Y represents H, (containing one crystal water) was obtained. Elemental analysis: C₁₃H₁₃N₃O₃.H₂O, Calculated values: C, 53.5%; H, 5.14%; N, 14.4%. Measured values: C, 54.19%; H, 4.75%; N, 14.5%. Water-solubility: 0.57 mg/ml (15° C.).

### Example 13

To 100 mL of anhydrous methanol was added a compound represented by formula (II), in which Y represents H, (518 mg). A solution of 98% concentrated sulfuric acid (200 mg) in anhydrous methanol (100 mL) was added. The mixture was stirred for 30 mins at room temperature to make the system clear. After the mixture was subjected to rotary evaporation under reduced pressure to remove the solvent and a solid was obtained, anhydrous THF was added and the mixture was stirred for further 1 hour, and then filtered with pump. The filter cake was dried for 10 hours in vacuo. A sulfate of Compound (II), in which Y represents H, (2:1) was obtained.

### Example 14

To 100 mL of anhydrous methanol was added a compound represented by formula (II), in which Y represents H, (518 mg). A solution of 1,5-naphthalene disulfonic acid (288 mg) in anhydrous methanol (100 mL) was added. The mixture was stirred for 30 mins at room temperature to make the system clear. After the mixture was subjected to rotary evaporation under reduced pressure to remove the solvent and a solid was obtained, anhydrous THF was added and the mixture was stirred for further 1 hour, and then filtered with pump. The filter cake was dried for 10 hours in vacuo. 1,5-naphthalene disulfonate of Compound (II), in which Y represents H, (2:1) was obtained.

### Example 15

To 30 mL of anhydrous ethanol was added a compound represented by formula (II), in which Y represents H, (518 mg). 0.27 mL of hydrobromic acid (40% aqueous solution) was added. The mixture was stirred at room temperature to make the system clear. After a solid precipitated slowly, the mixture was further stirred overnight and filtered with pump. The filter cake was dried for 10 hours in vacuo. A hydrobromide of Compound (II) (1:1) (containing one crystal water) was obtained. Elemental analysis: C₁₃H₁₃N₃O₃.HBr.1H₂O, Calculated values: C, 43.58%; H, 4.47%; N, 11.73%. Measured values: C, 43.24%; H, 4.70%; N, 11.68%. Melting point: decomposition at 217° C., Water-solubility: 5.5 mg/ml (15° C.).

### Reaction of a Compound Represented by Formula (II), in which Y Represents H, with a Weak Organic Acid

### Examples 16-26)

To 30 mL of anhydrous ethanol was added a compound represented by formula (II), in which Y represents H (518 mg) to obtain a solution of Compound (II), in which Y represents H, in anhydrous ethanol. 10 mL of anhydrous ethanol was added to an equal mole of a weak organic acid to give a solution. To the anhydrous ethanol solution of Compound (II), in which Y represents H, was added the solution of an organic acid in ethanol. The mixture was heated in an oil bath until reflux was happened. A small amount of water was added to make the system clear. After refluxing for 30 mins, the mixture was stirred at room temperature. After a solid precipitated, the mixture was stirred for further 4 hours, and then filtered with pump. After the filter cake was dried for 10 hours in vacuo, HNMR proved that the filter cake is the raw material (II), in which Y represents H (or the raw material (II), in which Y represents H, with one crystal water).

| Example | Organic acid |
|---|---|
| Example 16 | benzoic acid |
| Example 17 | succinic acid |
| Example 18 | fumaric acid |
| Example 19 | maleic acid |
| Example 20 | oxalic acid |
| Example 21 | glutamic acid |
| Example 22 | ascorbic acid |
| Example 23 | acetic acid |
| Example 24 | gluconic acid |
| Example 25 | salicylic acid |
| Example 26 | nicotinic acid |

### Example 27

### Stability of Aqueous Solution of 3-(4-amino-1-oxo-1,3-dihydro-isoindol-2-yl) piperidine-2,6-dione bisulfate tetrahydrate

### Example 7

17 mg of 3-(4-amino-1-oxo-1,3-dihydro-isoindol-2-yl)piperidine-2,6-dione bisulfate tetrahydrate (equivalent to 10 mg of 3-(4-amino-1-oxo-1,3-dihydro-isoindol-2-yl)piperidine-2,6-dione) was dissolved in 10 mL of deionized water and kept at room temperature for 12 hours. The content is higher than 98% of labeled amount.

### Example 28

### Preparation of Polymorph (IA)

To a 100 mL single-mouth bottle were added 550 mg of 3-(4-amino-1-oxo-1,3-dihydro-isoindol-2-yl)piperidine-2,6-dione and 30 mL of anhydrous ethanol. 1 mL of concentrated sulfuric acid was added under stirring into the mixture at room temperature. The resultant mixture was stirred overnight at room temperature and filtered with pump.

### Example 29

### Preparation of Polymorph (IB)

To a 250 mL single-mouth bottle were added 5.181 g of 3-(4-amino-1-oxo-1,3-dihydro-isoindol-2-yl)piperidine-2,6-dione and 150 mL of anhydrous ethanol. 2 mL of concentrated sulfuric acid was added under stirring into the mixture at room temperature. The resultant mixture was heated under reflux, and then stirred overnight at room temperature and filtered with pump.

### Example 30

### Preparation of Polymorph (IC)

To a 250 mL single-mouth bottle were added 2.59 g of 3-(4-amino-1-oxo-1,3-dihydro-isoindol-2-yl)piperidine-2,6-dione and 30 mL of acetone. 2.2 mL of concentrated sulfuric acid was dissolved in 20 mL of acetone and the mixture was dropwise added to the reaction bottle under stirring at room temperature. Subsequently, after stirring for 30 mins at 55° C., the mixture was stirred overnight at room temperature and filtered with pump.

### Example 31

### Preparation of Polymorph (IIA)

To a 100 mL single-mouth bottle were added 506 mg of 3-(4-amino-1-oxo-1,3-dihydro-isoindol-2-yl)piperidine-2,6-dione and 30 mL of anhydrous ethanol. 1 mL of concentrated nitric acid was added under stirring into the mixture at room temperature. The resultant mixture was stirred overnight at room temperature and filtered with pump.

### Example 32

### Preparation of Polymorph (IIIA)

To a 100 mL single-mouth bottle were added 518 mg of 3-(4-amino-1-oxo-1,3-dihydro-isoindol-2-yl)piperidine-2,6-dione and 30 mL of anhydrous ethanol. 378 mg of benzene sulfonic acid and 0.5 mL of water were added under stirring into the mixture at room temperature. The resultant mixture was heated under reflux for 30 mins, and then stirred overnight at room temperature and filtered with pump.

### Example 33

### Preparation of Polymorph (IIIB)

To a 500 mL single-mouth bottle were added 4.637 g of benzene sulfonic acid and 200 mL of ethanol, and then were added 6.473 g of 3-(4-amino-1-oxo-1,3-dihydro-isoindol-2-yl)piperidine-2,6-dione and 9 mL of water. The mixture was heated under reflux for 30 mins, and then stirred overnight at room temperature and filtered with pump.

### Example 34

### Preparation of Polymorph (IVA)

To a 100 mL single-mouth bottle were added 519 mg of 3-(4-amino-1-oxo-1,3-dihydro-isoindol-2-yl)piperidine-2,6-dione and 40 mL of anhydrous ethanol. 352 mg of p-toluene sulfonic acid and 1 mL of water were added under stirring into the mixture at room temperature. The resultant mixture was heated under reflux for 30 mins, and then stirred overnight at room temperature and filtered with pump.

### Example 35

### Preparation of Polymorph (VA)

To a 100 mL single-mouth bottle were added 519 mg of 3-(4-amino-1-oxo-1,3-dihydro-isoindol-2-yl)piperidine-2,6-dione and 40 mL of anhydrous ethanol. 0.27 mL of hydrobromic acid was added under stirring into the mixture at room temperature. The resultant mixture was stirred overnight at room temperature and filtered with pump.

### Example 36

### Preparation of Polymorph (VIA)

To a 250 mL single-mouth bottle were added 2.593 g of 3-(4-amino-1-oxo-1,3-dihydro-isoindol-2-yl)piperidine-2,6-dione and 70 mL of anhydrous ethanol. 0.78 mL of methylsulfonic acid was dissolved in a mixed solution of 10 mL of anhydrous ethanol and 1 mL of water, and then the mixture was dropwise added to the reaction bottle under stirring at room temperature. The resultant mixture was stirred overnight at room temperature and filtered with pump.

### Example 37

### Preparation of Polymorph (VIB)

To a 500 mL single-mouth bottle were added 6.472 g of 3-(4-amino-1-oxo-1,3-dihydro-isoindol-2-yl)piperidine-2,6-dione and 300 mL of anhydrous ethanol. 1.62 mL of methylsulfonic acid was added under stirring into the mixture at room temperature. The resultant mixture was heated under reflux for 30 mins, and then stirred overnight at room temperature and filtered with pump.

### Example 38

### Preparation of Polymorph (VIIA)

To a 250 mL single-mouth bottle were added 5.180 g of 3-(4-amino-1-oxo-1,3-dihydro-isoindol-2-yl)piperidine-2,6-dione and 150 mL of anhydrous ethanol. 1.6 mL of concentrated hydrochloric acid and 10 mL of water were added under stirring into the mixture at room temperature. The resultant mixture was stirred overnight at room temperature and filtered with pump.

### Example 39

### Preparation of Polymorph (VIIA)

To a 250 mL three-mouth bottle were added 1.038 g of 3-(4-amino-1-oxo-1,3-dihydro-isoindol-2-yl)piperidine-2,6-dione and 35 mL of tetrahydrofuran. Hydrogen chloride gas was introduced for 1 hour under stirring into the mixture at room temperature. The resultant mixture was stirred at room temperature and filtered with pump.

### Example 40

### Preparation of Polymorph (VIIA)

To a 100 mL single-mouth bottle were added 1.038 g of 3-(4-amino-1-oxo-1,3-dihydro-isoindol-2-yl)piperidine-2,6-dione and 30 mL of anhydrous ethanol. The mixture was heated to 60° C. 6 N of hydrochloric acid under stirring was added into the mixture until a clear solution was obtained. The temperature of the reaction solution was decreased to −20° C. The mixture was kept overnight and filtered with pump.

### Example 41

### Preparation of Polymorph (VIIA)

To a 100 mL single-mouth bottle were added 1.037 g of 3-(4-amino-1-oxo-1,3-dihydro-isoindol-2-yl)piperidine-2,6-dione and 30 mL of anhydrous ethanol. The mixture was heated to 60° C. 6 N of hydrochloric acid under stirring was added into the mixture until a clear solution was obtained. The mixture was kept overnight at room temperature and filtered with pump.

### Example 42

### Preparation of Polymorph (IVB)

To a 250 mL single-mouth bottle were added 5.18 g of 3-(4-amino-1-oxo-1,3-dihydro-isoindol-2-yl)piperidine-2,6-dione, 80 mL of anhydrous ethanol and 20 mL of water. The mixture was heated to 80° C., and then a clear solution was obtained. 4.18 g of p-toluene sulfonic acid was added into the mixture. The resultant mixture was stirred for further 3.5 hours and filtered with pump after the temperature was decreased to room temperature.

### Determination of Solubility:

### Determination of Saturated Solubility in Water of Sample in Each Example:

### Configuration of Standard Curve:

The sample in each example was scanned with UV spectroscopy in the wavelength range of 190-900 nm. The maximum absorbance was set to 2. All the samples to be determined have the same absorption peak at 304 nm. Although there are other absorption peaks in the range of 205-190 nm, the peak shapes are slightly different and the maximum absorption wavelengths thereof are different. Therefore, the fixed wavelength was determined to be 304 nm in the concentration versus absorbance determination.

During the preparation of saturated solutions, the solubility of the sample in Example 7 was found to be higher than 1 g/ml. Therefore, this compound was chosen to plot the concentration versus absorbance standard curve.

To a 100 mL volumetric flask were added the sample in Example 7 (279.26 mg) and distilled water. The volume was determined at 100 ml with distilled water. The concentration of the solution is 2.793 mg/mL. To a 50 mL volumetric flask was added 1 mL, 2 mL, 3 mL and 5 mL of the above solution, respectively. The volume was determined at 50 ml with distilled water. The concentrations of these solutions are 0.056 mg/mL, 0.112 mg/mL, 0.167 mg/mL and 0.279 mg/mL, respectively. The standard curve was plotted in the concentration versus absorbance coordinate system. γ2 coefficient is 0.99951 (see the Drawings of the Specification).

A small amount of the sample was used to prepare a 5 mL saturated solution. The solution was kept in a constant temperature water bath for 1 hour at 25° C. The supernatant was sampled and diluted to appropriate times according to the range of the standard curve. The concentration was determined by ultraviolet spectrophotometry, and the determined reading was multiplied by the dilution times to obtain the solubility of the sample to be determined.

1. A compound represented by formula (I) in polymorphic form, wherein XH represents a pharmaceutically acceptable strong acid, Y represents H, CH₃ or F, wherein the strong acid is an organic or inorganic acid of which pKa is less than pKa1 of phosphoric acid

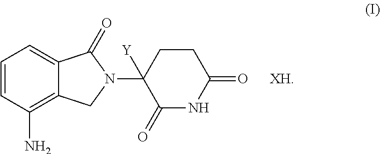

2. The compound represented by formula (I) in polymorphic form according to claim 1, wherein the strong acid is selected the group consisting of hydrochloric acid, hydrobromic acid, sulfuric acid, nitric acid and substituted sulfonic acid.

3. The compound represented by formula (I) in polymorphic form according to claim 2, wherein the substituted sulfonic acid is selected the group consisting of methylsulfonic acid, benzene sulfonic acid, p-toluene sulfonic acid, 1-naphthalenesulfonic acid, 2-naphthalenesulfonic acid, 1,5-naphthalene disulfonic acid and pyridinesulfonic acid.

4. The compound represented by formula (I) in polymorphic form according to claim 1, wherein the polymorphic compound is polymorph (IA) in which XH represents sulfuric acid and Y represents H, and which has X-ray powder diffraction spectra with the following parameters:

| Diffraction Angles (2θ, °) | Strength (I/I0) |
|---|---|
| 16.52 | 74.7 |
| 17.04 | 16.4 |
| 18.763 | 10.9 |
| 19.46 | 100 |
| 20.422 | 25.6 |
| 20.817 | 16.8 |
| 21.940 | 32.4 |
| 22.321 | 63.2 |
| 25.403 | 14.8 |
| 26.098 | 95.7 |
| 26.78 | 37.8 |
| 29.084 | 16.5 |
| 34.715 | 11.1 |

5. The compound represented by formula (I) in polymorphic form according to claim 1, wherein the polymorphic compound is polymorph (IB) in which XH represents sulfuric acid and Y represents H, and which has X-ray powder diffraction spectra with the following parameters:

| Diffraction Angles (2θ, °) | Strength (I/I0) |
|---|---|
| 5.580 | 53.6 |
| 10.437 | 17.3 |
| 12.820 | 50.0 |
| 16.759 | 79.6 |
| 17.139 | 19.6 |
| 18.860 | 100 |
| 19.241 | 38.2 |
| 20.641 | 54.6 |
| 21.099 | 37.3 |
| 21.700 | 22.5 |
| 22.759 | 21.0 |
| 24.158 | 14.4 |
| 25.118 | 15.8 |
| 26.739 | 67.9 |
| 27.359 | 40.1 |
| 30.020 | 13.6 |

6. The compound represented by formula (I) in polymorphic form according to claim 1, wherein the polymorphic compound is polymorph (IC) in which XH represents sulfuric acid and Y represents H, and which has X-ray powder diffraction spectra with the following parameters:

| Diffraction Angles (2θ, °) | Strength (I/I0) |
|---|---|
| 14.243 | 46.5 |
| 16.392 | 22.2 |
| 16.919 | 99.3 |
| 17.183 | 40.6 |
| 18.780 | 83.7 |
| 20.376 | 22.4 |
| 21.438 | 67.2 |
| 23.380 | 34.9 |
| 24.060 | 25.5 |
| 25.645 | 100 |
| 26.584 | 47.4 |
| 27.339 | 25.9 |
| 28.076 | 45.3 |
| 28.282 | 22.6 |
| 28.833 | 20.8 |
| 30.261 | 32.8 |
| 32.486 | 25.7 |

7. The compound represented by formula (I) in polymorphic form according to claim 1, wherein the polymorphic compound is polymorph (IIA) in which XH represents nitric acid and Y represents H, and which has X-ray powder diffraction spectra with the following parameters:

| Diffraction Angles (2θ, °) | Strength (I/I0) |
|---|---|
| 5.861 | 51.6 |
| 7.958 | 19.5 |
| 11.720 | 75.4 |
| 13.221 | 17.0 |
| 14.418 | 50.4 |
| 15.518 | 18.3 |
| 16.199 | 34.5 |
| 16.701 | 81.1 |
| 17.701 | 30.1 |
| 18.680 | 28.6 |
| 20.618 | 100 |
| 23.860 | 69.2 |
| 25.240 | 27.5 |
| 26.600 | 81.8 |
| 27.320 | 96.7 |
| 29.361 | 27.3 |

8. The compound represented by formula (I) in polymorphic form according to claim 1, wherein the polymorphic compound is polymorph (IIIA) in which XH represents benzene sulfonic acid and Y represents H, and which has X-ray powder diffraction spectra with the following parameters:

| Diffraction Angles (2θ, °) | Strength (I/I0) |
|---|---|
| 4.799 | 100 |
| 9.681 | 13.6 |
| 13.818 | 23.4 |
| 15.418 | 36.6 |
| 17.658 | 46.3 |
| 18.602 | 20.9 |
| 20.799 | 30.7 |
| 22.201 | 23.7 |
| 24.041 | 37.2 |
| 24.559 | 29.6 |
| 26.580 | 16.6 |

9. The compound represented by formula (I) in polymorphic form according to claim 1, wherein the polymorphic compound is polymorph (IIIB) in which XH represents benzene sulfonic acid and Y represents H, and which has X-ray powder diffraction spectra with the following parameters:

| Diffraction Angles (2θ, °) | Strength (I/I0) |
|---|---|
| 5.440 | 46.0 |
| 10.981 | 17.2 |
| 13.080 | 26.9 |
| 14.761 | 18.6 |
| 15.800 | 24.1 |
| 17.041 | 85.2 |
| 17.420 | 21.6 |
| 19.119 | 38.3 |
| 19.600 | 34.9 |
| 20.040 | 100 |
| 24.981 | 74.2 |
| 26.322 | 35.1 |
| 28.782 | 22.7 |

10. The compound represented by formula (I) in polymorphic form according to claim 1, wherein the polymorphic compound is polymorph (IVA) in which XH represents p-toluene sulfonic acid and Y represents H, and which has X-ray powder diffraction spectra with the following parameters:

| Diffraction Angles (2θ, °) | Strength (I/I0) |
|---|---|
| 4.640 | 100 |
| 13.580 | 32.3 |
| 14.360 | 17.1 |
| 15.200 | 21.1 |
| 17.399 | 59.5 |
| 19.339 | 15.7 |
| 19.938 | 18.8 |
| 20.560 | 24.3 |
| 24.181 | 68.0 |
| 27.583 | 15.3 |

11. The compound represented by formula (I) in polymorphic form according to claim 1, wherein the polymorphic compound is polymorph (IVB) in which XH represents p-toluene sulfonic acid and Y represents H, and which has X-ray powder diffraction spectra with the following parameters:

| Diffraction Angles (2θ, °) | Strength (I/I0) |
|---|---|
| 12.438 | 20.80 |
| 15.522 | 16.2 |
| 18.900 | 83.89 |
| 19.799 | 100 |
| 21.039 | 17.7 |
| 26.662 | 28.6 |

12. The compound represented by formula (I) in polymorphic form according to claim 1, wherein the polymorphic compound is polymorph (VA) in which XH represents hydrobromic acid and Y represents H, and which has X-ray powder diffraction spectra with the following parameters:

| Diffraction Angles (2θ, °) | Strength (I/I0) |
|---|---|
| 10.040 | 27.2 |
| 11.963 | 29.1 |
| 15.118 | 17.7 |
| 16.879 | 50.9 |
| 20.479 | 41.6 |
| 20.896 | 18.7 |
| 21.481 | 100 |
| 22.760 | 23.1 |
| 23.520 | 19.8 |
| 24.199 | 52.1 |
| 24.639 | 91.5 |
| 25.920 | 70.1 |
| 26.839 | 42.2 |
| 28.140 | 30.5 |
| 30.040 | 20.7 |
| 31.120 | 36.6 |
| 33.539 | 24.6 |
| 35.081 | 19.4 |

13. The compound represented by formula (I) in polymorphic form according to claim 1, wherein the polymorphic compound is polymorph (VIA) in which XH represents methylsulfonic acid and Y represents H, and which has X-ray powder diffraction spectra with the following parameters:

| Diffraction Angles (2θ, °) | Strength (I/I0) |
|---|---|
| 7.459 | 10.5 |
| 11.519 | 31.1 |
| 14.798 | 34.1 |
| 14.999 | 18.3 |
| 15.719 | 19.5 |
| 17.278 | 38.9 |
| 19.179 | 44.5 |
| 19.500 | 39.1 |
| 21.901 | 100 |
| 23.721 | 29.1 |
| 26.481 | 65.7 |
| 27.721 | 18.2 |

14. The compound represented by formula (I) in polymorphic form according to claim 1, wherein the polymorphic compound is polymorph (VIB) in which XH represents methylsulfonic acid and Y represents H, and which has X-ray powder diffraction spectra with the following parameters:

| Diffraction Angles (2θ, °) | Strength (I/I0) |
|---|---|
| 9.941 | 32.5 |
| 10.937 | 47.1 |
| 15.341 | 54.0 |
| 16.501 | 69.9 |
| 18.360 | 33.0 |
| 18.919 | 85.8 |
| 19.458 | 63.6 |
| 20.020 | 22.8 |
| 21.040 | 82.8 |
| 22.080 | 21.3 |
| 23.641 | 36.3 |
| 24.161 | 57.5 |
| 25.539 | 100 |
| 28.200 | 22.2 |
| 30.500 | 15.3 |
| 34.601 | 15.6 |

15. The compound represented by formula (I) in polymorphic form according to claim 1, wherein the polymorphic compound is polymorph (VIIA) in which XH represents hydrochloric acid and Y represents H, and which has X-ray powder diffraction spectra with the following parameters:

| Diffraction Angles (2θ, °) | Strength (I/I0) |
|---|---|
| 10.196 | 15.9 |
| 12.018 | 55.2 |
| 13.162 | 41.9 |
| 15.279 | 15.7 |
| 17.020 | 40.8 |
| 19.058 | 25.4 |
| 20.460 | 40.7 |
| 21.580 | 49.4 |
| 22.478 | 17.6 |
| 22.980 | 19.1 |
| 24.241 | 52.8 |
| 24.642 | 100 |
| 25.420 | 32.2 |
| 26.042 | 48.0 |
| 26.581 | 30.0 |
| 26.820 | 36.7 |
| 28.302 | 23.2 |
| 30.359 | 16.7 |
| 31.081 | 25.5 |
| 35.301 | 22.4 |

16. A pharmaceutical composition comprising a therapeutically effective amount of the compound represented by formula (I) in polymorphic form according to claim 1.

17. The pharmaceutical composition according to claim 16, wherein the pharmaceutical composition is formed in a tablet, a capsule, a powder injection, a solution formulation, a freeze-dried powder injection, an aerosol, a spray, a cream, a paste, eye drops, ear drops or an implant.

18. A process for preparing the compound represented by formula (I) in polymorphic form according to claim 1, comprising reacting a compound represented by formula (II) with an acid represented by XH in a suitable solvent system, 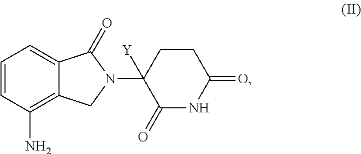
wherein XH represents a pharmaceutically acceptable strong acid, Y represents H, CH₃ or F.

19. A method for treating diseases or physiological abnormalities which can be cured by inhibiting inflammatory factors or angiogenesis in a subject, comprising administrating the subject with an therapeutically effective amount of the compound represented by formula (I) in polymorphic form according to claim 1.

20. The method according to claim 19, wherein the disease is selected from the group consisting of arthritis and cancer.

21. The method according to claim 20, wherein the disease is selected from the group consisting of hepatitis, gastritis, gastric ulcer, digestive ulcer, oral ulcer, nephritis, rhinitis, bronchitis, COPD, pneumonia, pulmonary tuberculosis, myocarditis, pancreatitis, prostatitis, cervicitis, enteritis, Crohn's syndrome, nerve endings inflammation, myelitis, encephalitis, peritonitis, Parkinson's disease, psoriasis, lupus erythematosus, refractory dermatitis and leprosy.

22. The method according to claim 19, wherein the cancer is selected from the group consisting of bone marrow cancer, leukemia, liver cancer, brain tumor, prostatic cancer, gastric cancer, esophagus cancer, intestine cancer, laryngeal cancer, oral cancer, nose cancer, bone cancer, cervical cancer, lung cancer, breast cancer, renal cancer, lymphoma, ovarian cancer, pancreatic cancer, adrenal cancer, mesothelial cell cancer, melanoma and myelodysplastic syndrome.
